(19) United States
(12) Patent Application Publication
(10) Pub. No.: US 2007/0078342 A1
(43) Pub. Date: Apr. 5, 2007

(54) Ultrasound imaging system and method having adaptive selection of image frame rate and/or number of echo samples averaged

(75) Inventor: James Jago, Seattle, WA (US)

Correspondence Address: PHILIPS MEDICAL SYSTEMS;PHILIPS INTELLECTUAL PROPERTY & STANDARDS, P.O. BOX 3003, 22100 BOTHELL EVERETT HIGHWAY, BOTHELL, WA, 98041-3003, US

(73) Assignee: Koninklijke Philips Electronics N.V., Eindhoven (NL)

(21) Appl. No.: 10/578,631

(22) PCT Filed: Nov. 4, 2004
(86) PCT No.: PCT/IB04/52314

§ 371 Date: May 9, 2006

Related U.S. Application Data
(60) Provisional application No. 60/524,406, filed on Nov. 21, 2003.

(51) Int. Cl. A61B 8/00

(52) U.S. Cl. 600/443

(57) ABSTRACT

An ultrasound diagnostic imaging system and method are described in which ultrasound image frames are generated by receiving ultrasound echo signals in response to respective ultrasound transmissions, sampling the echo signals, and then averaging the samples over a number of ultrasound transmissions. In one embodiment, the system allows a minimum image frame rate to be set, either directly by a user or indirectly from the rate of movement of physiological structures being imaged. The rate of movement of the structures is either estimated by the user or determined by the system.

This invention relates to ultrasound diagnostic imaging systems and, in particular, to ultrasound diagnostic imaging systems that have the ability to acquire ultrasound echo signals with adjustable signal averaging parameters and frame rate.

Ultrasound diagnostic imaging systems are in widespread use by cardiologists, obstetricians, radiologists and others for examinations of the heart, a developing fetus, internal abdominal organs and other anatomical structures. These systems operate by using an ultrasound transducer to transmit waves of ultrasound energy into the body, receiving ultrasound echoes reflected from tissue interfaces upon which the waves impinge, and translating the received echoes into corresponding echo signals. The echo signals generated by the transducer are then beamformed to focus the transmitted and received ultrasound into beams that may be steered in an azimuthal and/or elevational direction. After the received echo signals have been beamformed, they are processed to provide scan lines that are indicative of physiological structures positioned beneath a face of the transducer. A large number of scan lines are combined to produce an image frame from which an image of the physiological structures can be created.

The time required to create an image frame depends on the time required to transmit and receive ultrasound for the number of scan lines needed to form an image frame, and the time required to beamform and process received ultrasound echo signals to form the image frame. To a large extent, the minimum time required to acquire and create an image frame is fixed by the round trip transit time of ultrasound through the body to the physiological structures that are being imaged. Producing an ultrasound image of deeper structures requires that the ultrasound travel a greater round trip distance. The rate at which image frames can be created, known as the “frame rate,” will therefore be lower when imaging deeper structures.

While it is desirable to be able to image with a rapid frame rate, especially when imaging moving structures, it is also desirable in some procedures to be able to penetrate to and clearly image structures at considerable depth. But the depth of penetration can be limited by factors such as the frequency of the transmitted or received ultrasound, which attenuates with passage through tissue. The dynamic range of the ultrasound system may also provide an impediment to imaging at considerable depths, and the attenuation of ultrasound by the target structure may also limit penetration.

One way to improve the clarity of images from considerable depths, but at the expense of the ultrasound frame rate, is to generate scan lines by averaging echo signals from multiple ultrasound transmissions. Signal averaging is a technique that can minimize the effect of signal noise. This technique involves rapidly obtaining multiple samples of the same signal, each of which can be thought of as an estimate of the true value of the signal in the absence of noise. These samples are then averaged to improve the signal-to-noise ratio. In ultrasound imaging, signal averaging has the benefit of increasing the depth at which physiological structures can be imaged. More specifically, for fully random noise and fully correlated signals (i.e., signals that do not change between samples), the signal-to-noise ratio will improve with the square root of N, where N is the number of samples of the signal. For ultrasound imaging, an increase AD in the depth at which imaging can be achieved can be calculated using the following equation:

$$Δ D = \frac{20 log 10 ( \sqrt{N} )}{{F}_{C} μ}$$

Where: N is the number of samples being averaged
Fc is the imaging frequency (MHz)
μ is the round-trip attenuation (dB/cm/MHz)

For an ultrasound transducer operating at approximately 3 MHz in a medium with a round-trip attenuation of 0.6 dB/cm/Mhz, which is about average for soft tissue, averaging ultrasound echo signals over four ultrasound transmissions would provide more than about 3 cm of additional imaging depth. However, requiring four ultrasound transmissions for each image frame would also decrease the frame rate by a factor of four. As a result, the operation of all ultrasound imaging systems results in a compromise between imaging depth and frame rate.

The trade-off between imaging depth and frame rate is usually determined by the ultrasound system in response to the selection by the sonographer of different imaging parameters. For example, the sonographer can select a desired probe frequency, harmonic or fundamental frequency operation, and the depth and number of focal zones, among other parameters. These selections then lead to a determination of the frame rate or the number of samples that can be averaged for noise performance improvement. However, the user is generally not aware of exactly how the maximum frame rate or number of samples is affected by his or her selection of these parameters. As a result, it is often difficult to arrive at both a desirable frame rate and sample averaging which enables penetration to a specific depth. It is also at times difficult to select a variety of parameters that will allow a frame rate sufficient to image moving structures.

The above-described uncertainties make it difficult for a sonographer to optimally select either the frame rate or the number of samples to average. If a sonographer needs to image deep physiological structures and therefore would like a large number of samples to be averaged, the sonographer will be unaware of the degree to which that selection may adversely affect viewing the movement of such structures. Similarly, if a sonographer needs to image rapidly moving physiological structures and therefore makes selections leading to a high frame rate, the sonographer may be unaware of the degree to which those selections may adversely affects the ability to clearly image such structures if they are relatively deep.

There is therefore a need for an ultrasound imaging system and method that allows easier and more optimum selection of the number of samples that can be averaged to improve image clarity at deeper depths within the constraint of an acceptable frame rate of display.

The present invention is a system and method for generating an ultrasound image by repetitively transmitting ultrasound into a region of interest and receiving ultrasound echo signals resulting from each of the transmissions. The ultrasound echo signals are sampled to provide echo signal samples, and ultrasound image frames are generated by averaging corresponding echo signal samples over a number of ultrasound transmissions. The image frames are then used to create a displayed ultrasound image.

The image frames are generated at a frame rate that is a function of the number of transmissions over which the echo signal samples are averaged. Averaging echo signal samples over a greater number of ultrasound transmissions requires a greater amount of time, and therefore reduces the frame rate. The image frame rate and sample averaging number are determined in accordance with the invention using a minimum frame rate criterion and calculating the number of transmissions over which the echo signal samples could be averaged to achieve that frame rate. The minimum frame rate may be entered directly by a user or it may be determined based on the type of ultrasound examination being conducted or the rate at which imaged physiological structures are moving or are expected to move.

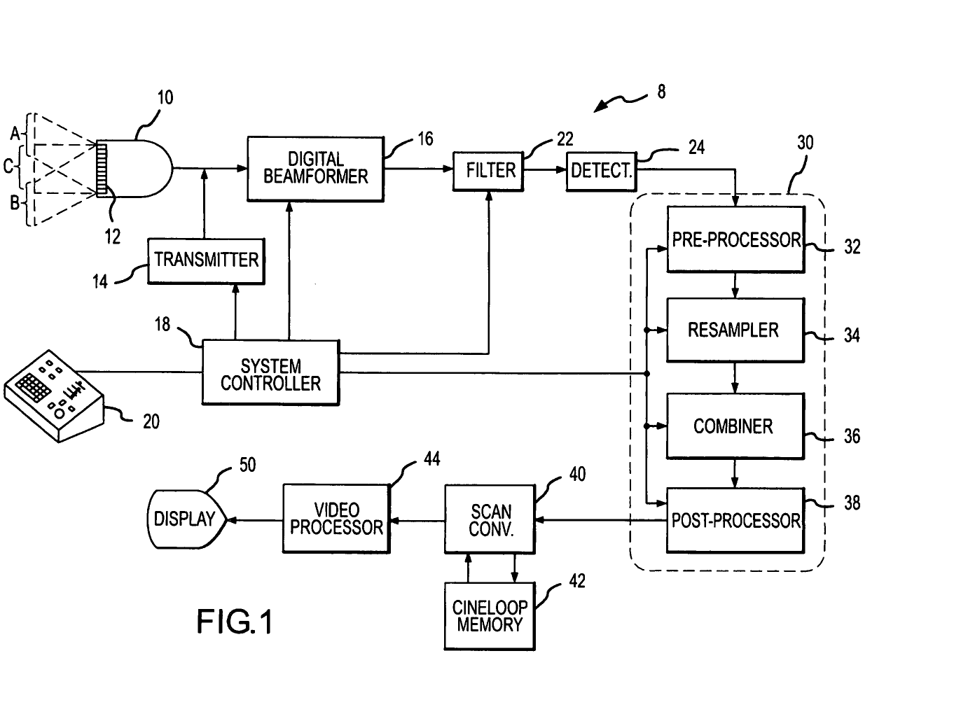

FIG. 1 is a block diagram of an ultrasound imaging system according to one embodiment of the invention.

One embodiment of an ultrasound diagnostic imaging system 8 according to the present invention is shown in FIG. 1. However, it will be understood that other imaging systems can be used in place of the imaging system 8 shown in FIG. 1, as will be apparent to those skilled in the art. Accordingly, the drawings and detailed description are to be regarded as illustrative in nature and not restrictive.

The imaging system 8 includes a scanhead 10 having an array transducer 12 that transmits beams of ultrasound at different angles over an image field denoted by the dashed rectangle and parallelograms. Three groups of scanlines are indicated in the drawing, labeled A, B, and C, with each group being steered at a different angle relative to the scanhead 10.

The transmission of the beams is controlled by a transmitter 14, which controls the phasing and time of actuation of each of the elements of the array transducer 12 so as to transmit each beam from a predetermined origin along the array and at a predetermined angle. The echoes returned from along each scanline are received by individual elements (not shown) of the array transducer 12 and coupled to a digital beamformer 16. The beamformer 16 repetitively samples each of the signals, and converts each sample to a digitized sample using a conventional analog-to-digital converter in the beamformer 16. The digital beamformer 16 digitally processes the samples to effectively delay and sum the echoes from the elements in the array transducer 12 to form a sequence of focused, coherent digital echo samples along each scanline.

The transmitter 14 and beamformer 16 are operated under control of a system controller 18, which is, in turn, responsive to the settings of controls on a user interface 20 operated by a user of the ultrasound system. The user interface 20 also allows the user to enter a value for the minimum frame rate that can be tolerated, the minimum number of samples that should be averaged, the imaging depth, the rate of image movement, and/or the type of examination being conducted, which can be used to determine the value of one of the foregoing parameters. The system controller 18 controls the transmitter 14 to transmit the desired number of scanline groups at the desired angles, transmit energies and frequencies. The system controller 18 also controls the digital beamformer 16 to properly delay and combine the received echo signals for the apertures and image depths used.

The scanline echo signal samples are filtered by a programmable digital filter 22, which defines the band of frequencies of interest. When imaging harmonic contrast agents or performing tissue harmonic imaging, the passband of the filter 22 is set to pass harmonics of the transmit band. The filtered signals are then detected by a detector 24. In a preferred embodiment the filter 22 and detector 24 include multiple filters and detectors so that the received signals may be separated into multiple passbands, individually detected and recombined to reduce image speckle by frequency compounding. For B mode imaging the detector 24 will perform amplitude detection of the echo signal envelope. For Doppler imaging, ensembles of echoes are assembled for each point in the image and are Doppler processed to estimate the Doppler shift or Doppler power intensity.

The digital echo signals are then processed in a processor 30. If spatial compounding is used, the processor also performs spatial compounding processing. The digital echo signals are initially pre-processed by a preprocessor 32. The pre-processor 32 can preweight the signal samples if desired with a weighting factor. The samples can be preweighted with a weighting factor that is a function of the number of image frames used to form a particular compound image. The pre-processor 32 can also weight edge lines that are at the edge of one overlapping image so as to smooth the transitions where the number of samples or images which are compounded changes. The pre-processed signal samples may then undergo a resampling in a resampler 34. The resampler 34 can spatially realign the estimates of one component frame to those of another component frame or to the pixels of the display space.

After resampling, the image frames may be compounded by a combiner 36. Combining may comprise summation, averaging, peak detection, or other combinational means. The samples being combined may also be weighted prior to combining in this step of the process. Finally, post-processing is performed by a post-processor 38. The post-processor normalizes the combined values to a display range of values. Post-processing can be most easily implemented by look-up tables and can simultaneously perform compression and mapping of the range of compounded values to a range of values suitable for display of the compounded image.

Scan conversion is subsequently performed by a scan converter 40. The compound images may be stored in a Cineloop memory 42 in either estimate or display pixel form. If stored in estimate form, the images may be scan converted when replayed from the Cineloop memory for display. The scan converter 40 and Cineloop memory 42 may also be used to render three dimensional presentations of the images, as described in U.S. Pat. Nos. 5,485,842 and 5,860,924, or display of an extended field of view by overlaying successively acquired, partially overlapping images in the lateral dimension. Following scan conversion, the images are processed for display by a video processor 44 and displayed on an image display 50.

In accordance with one embodiment of the present invention, the system controller 18 also controls the imaging system 8 based on a value entered through the user interface 20 for the minimum frame rate that can be tolerated or the minimum number of samples that should be averaged. Alternatively, a user may enter information via the user interface 20 that allows the system controller 18 to determine either a frame rate or the number of samples that should be averaged. For example, the user may enter a value for the depth to which imaging will be performed, which would allow the system controller 18 to determine a suitable value for the number of samples that will be averaged. Similarly, the user may enter a value for the rate that tissues are expected to be moving based on the type of physiological structure being imaged, which would allow the system controller 18 to determine a suitable value for the frame rate. Instead of entering those values directly, the user may enter information about the type of examination being conducted, which would allow the system controller 18 to determine either the image frame rate, number of samples to be averaged, or a combination of image frame rate and number of samples to be averaged. For example, the user may indicate that a cardiac ultrasound examination is to be conducted. The system controller 18 will then select a frame rate that is high enough to accommodate the movement at the heart, and it will set a sample average number that is sufficiently high to allow sampling at the depth of the heart. Other operating alternatives will be apparent to one skilled in the art.

In one embodiment of the invention, a value for a minimum acceptable frame rate FR_{MIN} is entered by the user, and the number of pulses N that are to be averaged is calculated by the following formula:
N=(frame rate achievable with no sample averaging)/FR_{MIN}

For example, if a value of 10 frames/sec. is entered for FR_{MIN}, and the frame rate achievable by the system 8 is 90 frames/sec., then the system controller 18 will calculate a value of 9 for N using the above formula. The system 8 will therefore average samples from 9 ultrasound transmission for each image frame. In another embodiment of the invention, rather than entering a value for the minimum acceptable frame rate FR_{MIN}, the user may enter information about the rate at which the image tissue is expected to be moving or the type of examination to be conducted, and the system controller 18 will calculate the minimum acceptable frame rate FR_{MIN} based on that information.

The foregoing example assumes that the frame size and line density is unchanged. An alternative approach is to vary the frame characteristics to enable the frame to be acquired in less time. For example, the initial frame may be a sector image of 90°. Narrowing the sector width to a lesser dimension such as 30o will decrease the time needed to scan the image area. Thus, the system can diminish the sector angle to maintain the frame rate above the minimum and still enable the acquisition of multiple samples along each scanline for averaging to improve penetration. The system can present a suggested narrower sector width outline on top of the initial sector, enabling the user to select the narrower width and, if desired, to position the narrower sector so that it is centered on the anatomy of interest.

In another embodiment of the invention, the user may enter information about the type of examination to be conducted, and the system controller 18 will determine the optimum tradeoff between frame rate and the number of samples that are to be averaged. For example, the user may indicate that a cardiac ultrasound examination is to be conducted. The system controller 18 will then determine, based on the expected rate of movement of the heart and the depth of the heart beneath the skin, that a frame rate of 18 frames/sec. should be used and 5 samples should be averaged. Other means of determining the frame rate and number of pulses averaged will be apparent to one skilled in the art.

In still another embodiment of the invention, the frame rate and sample averaging number are optimized by the system controller 18 based on the characteristics of the generated ultrasound image and the manner in which it is being obtained. More specifically, the system controller 18 selects a desired sample averaging number based on the depth to which physiological structures are being scanned. An ultrasound image is then produced and analyzed by the processor 30 to determine the rate at which portions of the image move from frame-to-frame. A variety of techniques known to one skilled in the art can be used to determine frame-to-frame movement. Based on the determined frame-to-frame movement, the processor 30 or the system controller 18 selects a desired frame rate. The system controller 18 then selects a final frame rate and sample average number based on a compromise between the trade-offs between achieving the desired frame rate and the desired sample average number. If desired, the processor 30 and system controller 18 can perform several iterations of examining the image from frame-to-frame and then adjusting the frame rate and sample average number. The ultrasound imaging system and method is therefore able to adapt itself to the optimum compromise between frame rate and signal averaging number with minimal or no user input.

From the foregoing it will be appreciated that, although specific embodiments of the invention have been described herein for purposes of illustration, persons skilled in the art will recognize that various modifications may be made without deviating from the spirit and scope of the invention. Accordingly, the invention is not limited except as by the appended claims.

What is claimed is:

1. A method of generating an ultrasound image, comprising:
repetitively transmitting ultrasound into a region of interest;
receiving ultrasound echo signals resulting from each of the transmissions;
sampling the ultrasound echo signals to provide echo signal samples;
setting a minimum value of a first operating parameter, the first operating parameter being a frame rate at which the image frames are to be created;
determining a value for a second operating parameter that is different from the first operating parameter based on a minimum value of the first operating parameter, the second operating parameter being the number of transmissions over which the echo signal samples are to be averaged to create the ultrasound image frames;
creating ultrasound image frames using the minimum value of the first operating parameter and the determined value of the second operating parameter; and
displaying an ultrasound image using the created ultrasound image frames.

2. The method of claim 1 wherein the acts of setting a minimum value of the first operating parameter and determining a value for the second operating parameter comprise setting a minimum value for the ultrasound frame rate and determining a value for the number of transmissions over which the echo signal samples are to be averaged to create the ultrasound image frames based on the minimum value for the ultrasound frame rate.

3. The method of claim 2 wherein the act of determining the number of transmissions over which the echo signal samples are to be averaged to create the ultrasound image frames comprises determining the number of transmissions over which the echo signal samples are to be averaged based on the formula N=FR_{MAX}/FR_{MIN}, where FR_{MAX} is the maximum frame rate that can be achieved without averaging the echo signal samples over multiple transmissions, FR_{MIN} is the set minimum value for the ultrasound frame rate, and N is the determined number of transmissions over which the echo signal samples are to be averaged.

4. The method of claim 2 wherein the act of setting a minimum value for the ultrasound frame rate comprises estimating the rate of movement of physiological structures in the region of interest, and setting the minimum value for the ultrasound frame rate as a function of the estimated rate of movement of the physiological structures.

5. The method of claim 2 wherein the act of setting a minimum value for the ultrasound frame rate comprises:
creating a plurality of ultrasound image frames;
analyzing the ultrasound image frames to determine the frame-to-frame changes corresponding to movement of imaged physiological structures; and
setting the minimum value for the ultrasound frame rate as a function of the ultrasound image frame analysis.

6. The method of claim 2 wherein the act of setting a minimum value for the ultrasound frame rate comprises providing information about a type of ultrasound examination that will be conducted, and setting the minimum value for the ultrasound frame rate as a function of the type of ultrasound examination that will be conducted.

7. A method of generating an ultrasound image, comprising:
repetitively transmitting ultrasound into a region of interest;
receiving ultrasound echo signals resulting from each of the transmissions;
sampling the ultrasound echo signals to provide echo signal samples;
creating a plurality of ultrasound image frames using a preliminary value for a frame rate at which the ultrasound image frames will be created and a preliminary value for the number of transmissions over which the echo signal samples are to be averaged to create the ultrasound image frames;
analyzing the ultrasound image frames or the manner in which the ultrasound image frames were created using the preliminary values;
based on the analysis, determining a final value for the frame rate and a final value for the number of transmissions over which the echo signal samples are to be averaged to create the ultrasound image frames;
creating ultrasound image frames using the final value for the frame rate and the final value for the number of transmissions over which the echo signal samples are to be averaged to create the ultrasound image frames; and
displaying an ultrasound image using the created ultrasound image frames.

8. The method of claim 7 wherein the act of analyzing the ultrasound image frames or the manner in which the ultrasound image frames were created comprises analyzing the ultrasound image frames that were created using the preliminary values to determine the frame-to-frame changes corresponding to movement of imaged physiological structures.

9. The method of claim 7 wherein the act of analyzing the ultrasound image frames or the manner in which the ultrasound image frames were created comprises determining the depth to which physiological structures in the region of interest will be imaged.

10. The method of claim 7 wherein the act of analyzing the ultrasound image frames or the manner in which the ultrasound image frames were created comprises determining a narrower sector for image frames.11. An ultrasound diagnostic imaging system, comprising:
a ultrasound scanhead including an array transducer;
an ultrasound transmitter coupled to the array transducer in the scanhead to apply transmit signals to the array transducer;
a controller coupled to the transmitter, the controller being operable to trigger the ultrasound transmitter to repetitively apply transmit signals to the array transducer thereby causing the array transducer in the scan head to transmit ultrasound into a region of interest, the controller further receiving a minimum value of a first operating parameter, the first operating parameter being a frame rate at which the image frames are to be created, the controller further determining a value for a second operating parameter that is different from the first operating parameter based on a minimum value of the first operating parameter, the second operating parameter being the number of transmissions over which the echo signal samples are to be averaged to create the ultrasound image frames or the frame rate at which the image frames are to be created;
a beamformer coupled to the controller and to the array transducer in the scanhead to receiving ultrasound echo signals resulting from each of the transmissions and form the received ultrasound echo signals into beams;
a processor coupled to the beamformer, the processor being operable to create ultrasound image frames using the minimum value of the first operating parameter and the determined value of the second operating parameter; and
a display coupled to the processor, the processor being operable to display an ultrasound image using the created ultrasound image frames.

12. The ultrasound diagnostic imaging system of claim 11, further comprising a user interface coupled to the controller, the user interface being operable to allow a user to enter a minimum value of the first operating parameter.

13. The ultrasound diagnostic imaging system of claim 12 wherein the minimum value of the first operating parameter entered into the user interface comprises a minimum value for the ultrasound frame rate.

14. The ultrasound diagnostic imaging system of claim 13 wherein the controller is operable to determine a value N for the number of transmissions over which the echo signal samples are to be averaged based on the formula N=FR_{MAX}/FR_{MIN}, where FR_{MAX} is the maximum frame rate that can be achieved without averaging the echo signal samples over multiple transmissions, and FR_{MIN} is the set minimum value for the ultrasound frame rate.

15. The ultrasound diagnostic imaging system of claim 11, further comprising a user interface coupled to the controller, the user interface being operable to allow a user to enter an estimate of the rate of movement of physiological structures in the region of interest, and wherein the controller is operable to set the minimum value for the ultrasound frame rate as a function of the estimated rate of movement of the physiological structures.

16. The ultrasound diagnostic imaging system of claim 11, further comprising a user interface coupled to the controller, the user interface being operable to allow a user to enter information about a type of ultrasound examination that will be conducted.

17. The ultrasound diagnostic imaging system of claim 16 wherein the controller is operable to set the minimum value for the ultrasound frame rate as a function of the type of ultrasound examination that will be conducted.

18. The ultrasound diagnostic imaging system of claim 11, further comprising a user interface coupled to the controller, the user interface being operable to allow a user to enter an estimate of the depth to which physiological structures in the region of interest will be imaged, and wherein the controller is operable to set the minimum value for the number of transmissions over which the echo signal samples are to be averaged as a function of the estimated depth to which physiological structures in the region of interest will be imaged.
